(19) United States
(12) Patent Application Publication
(10) Pub. No.: US 2025/0210170 A1
(43) Pub. Date: Jun. 26, 2025

(54) IDENTIFYING PERTURBATIONS TO CONTROL CELLULAR STATES WITH GENERATIVE ADVERSARIAL NETWORKS

(71) Applicant: KOREA ADVANCED INSTITUTE OF SCIENCE AND TECHNOLOGY, Daejeon (KR)

(72) Inventors: Kwang-Hyun CHO, Daejeon (KR); Younghyun HAN, Daejeon (KR)

(21) Appl. No.: 18/986,472

(22) Filed: Dec. 18, 2024

(30) Foreign Application Priority Data
Dec. 26, 2023 (KR) 10-2023-0191567

(51) Int. Cl. G16H 20/10; G06N 3/0455; G16H 50/20

(52) U.S. Cl. CPC G16H 20/10; G06N 3/0455; G16H 50/20

(57) ABSTRACT

A method for determining suitability of a drug administered for cell state transition, including: acquiring baseline states of actual cells, perturbed states of the actual cells to which the drug is administered, an initial cell state of a patient, and a target cell state representing the normal state of the patient's cells; encoding the baseline states, the perturbed states, the initial cell state, and the target cell state to generate latent baseline states, latent perturbed states, a latent initial cell state, and a latent target cell state, respectively; calculating a representative distance vector indicating distance between the latent baseline states and the latent perturbed states, and calculating a reference distance vector, which is the distance vector between the latent initial cell state and the latent target cell state; and determining suitability of the drug based on the degree of similarity between the representative distance vector and the reference distance vector.

### CROSS-REFERENCE TO RELATED APPLICATION

The priority of Republic of Korea Patent Application 10-2023-0191567 filed Dec. 26, 2023 is hereby claimed under the provisions of 35 USC § 119. The disclosure of Korea Patent Application 10-2023-0191567 is hereby incorporated herein by reference, in its entirety, for all purposes.

### TECHNICAL FIELD

The present invention relates to a technology that uses computer simulation to predict the perturbation response caused by a drug administered to cells and determines the target drug that transitions the state of a cell to a desired state.

### BACKGROUND TECHNOLOGIES

The present invention relates to a technology for altering the state of a cell, where the state of a cell can be defined based on the expression levels of biomolecules expressed within the cell. These expression levels can vary over time. Assuming that the number of biomolecules predominantly expressed within the cell is M, an array consisting of M numerical values representing the expression levels of these M genes in the cell can be defined as the state of a cell. The expression levels of these M genes within the cell may be obtained using well-known sequencing techniques. The space composed of the states of cells observed from multiple cells can be referred to as the cell state space.

The present invention proposes a method for encoding the cell state in the cell state space into a latent state in the latent space to find the optimal drug for changing the first state of a cell to a second state. In this context, the encoder that performs this encoding can be generated using a GAN (Generative Adversarial Network). A GAN is a well-known type of neural network that includes a generator, which transforms data belonging to the latent space to generate fake information, and a discriminator, which determines whether the generated information is real or fake. The states obtained as a result of transforming the cell states existing in the cell state space using the encoder are referred to as latent states, and the space composed of various latent states can be referred to as the latent space.

### DETAILED DESCRIPTION OF THE INVENTION

### Technical Problems

The present invention aims to provide a GAN model that can simulate perturbations through arithmetic operations within the latent space. Furthermore, the present invention aims to provide a technology for finding the optimal perturbations to transition the state of a cell represented within the latent space to a desired state.

### Technical Solutions

According to one aspect of the invention, a method for determining the suitability of an administered drug for cell state transition, which determines the suitability of the k-th drug administered to transition the state of a cell to another state, can be provided. This method includes the steps of: acquiring, by a computing apparatus, a set of baseline states 101, which are data representing states of a set of actual cells, acquiring a k-th set of perturbed states 102, which are data representing transitioned states of the set of actual cells after administering the k-th drug; acquiring an initial cell state 103, which is data representing an initial state of a specific cell line of a first subject; and acquiring a target cell state 104, which is data representing a target state of the specific cell line of the first subject; encoding, by the computing apparatus, the set of baseline states 101, the k-th set of perturbed states 102, the initial cell state 103, and the target cell state 104 to generate a set of latent baseline states 201, a k-th set of latent perturbed states 202, a latent initial cell state 203, and a latent target cell state 204, respectively; calculating, by the computing apparatus, a k-th representative distance vector that indicates a distance between the set of latent baseline states and the k-th set of latent perturbed states, and calculating a reference distance vector (DR), which is a distance vector between the latent initial cell state and the latent target cell state; and calculating, by the computing apparatus, a degree of similarity between the k-th representative distance vector and the reference distance vector to determine suitability of the k-th drug based on the calculated degree of similarity

According to another aspect of the invention, a computing apparatus comprising: a processing unit and a storage unit may be provided. The storage unit contains encoder instruction codes that execute a predetermined encoder and suitability determination instruction codes that execute a method for determining suitability of a k-th drug administered to transition the state of a cell to another state. Furthermore, the processing unit, by reading and executing the suitability determination instruction codes from the storage unit, performs the steps of: acquiring a set of baseline states, which are data representing states of a set of actual cells, acquiring a k-th set of perturbed states, which are data representing transitioned states of the set of actual cells after administering the k-th drug; acquiring an initial cell state, which is data representing an initial state of a specific cell line of a first subject; and acquiring a target cell state, which is data representing a target state of the specific cell line of the first subject; encoding the set of baseline states, the k-th set of perturbed states, the initial cell state, and the target cell state 104 to generate a set of latent baseline states, a k-th set of latent perturbed states, a latent initial cell state, and a latent target cell state, respectively; calculating a k-th representative distance vector that indicates a distance between the set of latent baseline states and the k-th set of latent perturbed states, and calculating a reference distance vector, which is a distance vector between the latent initial cell state and the latent target cell state; and calculating a degree of similarity between the k-th representative distance vector and the reference distance vector to determine suitability of the k-th drug based on the calculated degree of similarity.

Here, the processing unit may be configured to independently and repeatedly execute the suitability determination instruction codes for multiple different administered drugs and to determine the suitability ranking of the multiple administered drugs with reference to the multiple suitabilities determined for the multiple different administered drugs.

According to another aspect of the invention, a non-volatile storage medium readable by a computing apparatus which stores a software program may be provided. The software program may records suitability determination instruction codes for executing a method for determining suitability of a k-th drug administered to transition a state of a cell to another state. The suitability determination instruction codes may allow the computing apparatus to perform the steps of acquiring a set of baseline states, which are data representing states of a set of actual cells, acquiring a k-th set of perturbed states, which are data representing transitioned states of the set of actual cells after administering the k-th drug; acquiring an initial cell state, which is data representing an initial state of a specific cell line of a first subject; and acquiring a target cell state, which is data representing a target state of the specific cell line of the first subject; encoding the set of baseline states, the k-th set of perturbed states, the initial cell state, and the target cell state 104 to generate a set of latent baseline states, a k-th set of latent perturbed states, a latent initial cell state, and a latent target cell state, respectively; calculating a k-th representative distance vector that indicates a distance between the set of latent baseline states and the k-th set of latent perturbed states, and calculating a reference distance vector, which is a distance vector between the latent initial cell state and the latent target cell state; and calculating a degree of similarity between the k-th representative distance vector and the reference distance vector to determine suitability of the k-th drug based on the calculated degree of similarity.

Here, the software program my include encoder training instruction codes which implement an encoding training unit configured to train an encoder performing the encoding. The encoder training unit

The software program further includes encoder training instruction codes to execute an encoder training unit that trains the encoder performing the encoding. The encoder training unit may be configured to train a Variational AutoEncoder (VAE), which includes an encoder and a decoder, and a GAN, which includes a generator and a discriminator. The generator may be the decoder, and the generator may be configured to receive output values from the encoder. The encoder training instruction codes may instruct the computing apparatus to input a state of an arbitrary cell into the encoder to output a latent state, the output latent state is input into the generator to output a reconstructed state, and allow the discriminator to determine the truthfulness of the outputted reconstructed state based on the outputted reconstructed state and the state of the arbitrary cell. This way, the encoder, the generator, and the discriminator may be trained accordingly.

Here, in the method for determining the suitability of the administered drug for cell state transition, the computing apparatus, and the non-volatile storage medium, the higher the degree of similarity, the higher the suitability value can be.

Here, in the method for determining the suitability of an administered drug for cell state transition, the computing apparatus, and the non-volatile storage medium, the encoder used by the computing apparatus for encoding may be the encoder of a Variational AutoEncoder (VAE) trained using a specific encoder training unit executed by the computing apparatus. The encoder training unit may include a Generative Adversarial Network (GAN) that comprises a generator and a discriminator. The generator may be the decoder of the VAE, and the values output by the VAE's encoder may be input into the generator. The encoder training unit may be configured to train the encoder, generator, and discriminator such that the state of an arbitrary cell is input into the encoder to output a latent state, the output latent state is input into the generator to output a reconstructed state, and allow the discriminator to determine the truthfulness of the outputted reconstructed state based on the outputted reconstructed state and the state of the arbitrary cell. This way, the encoder, the generator, and the discriminator may be trained accordingly.

Here, the method for determining the suitability of an administered drug for cell state transition, the computing apparatus, and the non-volatile storage medium can be such that the method by which the encoder training unit trains the encoder includes the steps of: acquiring, by the computing apparatus, a first-first perturbed state x_{Aα}, which is a state of a cell of a first cell line A after administering the first drug α; a first-second perturbed state x_{Bα}, which is a state of a cell of a second cell line B after administering the first drug α; a second-second perturbed state x_{Bβ}, which is a state of the cell of the second cell line after administering a second drug β; and a second-first perturbed state x_{Aβ}, which is a state of the cell of the first cell line (A) after administering the second drug; independently inputting, by the computing apparatus, the first-fist perturbed state x_{Aα}, the first-second perturbed state x_{Bα}, and the second-second perturbed state x_{Bβ} into the encoder to generate a first-first latent perturbed state z_{Aα}, a first-second latent perturbed state z_{Bα}, and a second-second latent perturbed state z_{Bβ}, respectively; inputting, by the computing apparatus, a latent state z′_{Aβ}, which is obtained by adding the second-second latent perturbed state z_{Bβ} to a value obtained by subtracting the first-second latent perturbed state z_{Ba} from the first-first latent perturbed state z_{Aα}, into the generator to generate a second-first reconstructed perturbed state x′_{Aβ}; and training, by the computing apparatus, the encoder using a first loss Lₜᵣᵢₚₗₑ, which utilizes a value obtained by subtracting the second-first perturbed state x_{Aβ} from the second-first reconstructed perturbed state x′_{Aβ}. Here, the encoder training instruction codes may include a first set of instruction codes to execute the aforementioned steps.

Here, the method for determining the suitability of an administered drug for cell state transition, and training the computing apparatus, and the non-volatile storage medium can be such that the method by which the encoder training unit trains the encoder may include the steps of acquiring, by the computing apparatus, a first-first perturbed state x_{Aα}, which is a state of a cell of a first cell line A after administering the first drug α; a first cell state x_{A}, which is a state of a cell of the first cell line, a first-second perturbed state x_{Bα}, which is a state of a cell of a second cell line B after administering the first drug α, and a second cell state x_{B}, which is a state of a cell of the second cell line; independently inputting, by the computing apparatus, the first-first perturbed state x_{Aα}, the first cell state x_{A}, the first-second perturbed state x_{Bα}, and the second cell state x_{B} into the encoder to generate a first-first latent perturbed state z_{Aα}, a first latent cell state z_{A}, a first-second latent perturbed state z_{Bα}, and a second latent cell state z_{B}, respectively; generating, by the computing apparatus, a first perturbation vector z^{A}_{α} by subtracting the first latent cell state z_{A} from the first-first latent perturbed state z_{Aα}; and generating, by the computing apparatus, a second perturbation vector z^{B}_{α} by subtracting the second latent cell state z_{B} from the first-second latent perturbed state z_{Bα}; and training, by the computing apparatus, the encoder using a second loss L_{delta}, which utilizes a value obtained by subtracting the second perturbation vector z^{B}_{α} from the first perturbation vector z^{A}_{α}. Here, the encoder training instruction codes may include a second set of instruction codes to execute the aforementioned steps.

Here, the method for determining the suitability of an administered drug for cell state transition, and training the computing apparatus, and the non-volatile storage medium may be such that the method by which the encoder training unit trains the encoder may include the steps of acquiring, by the computing apparatus, a first-first perturbed state x_{Aα}, which is a state of a cell of the first cell line A after administering the first drug α, a first-second perturbed state x_{Ba} which is a state of a cell of a second cell line B after administering the first drug α; a second-second perturbed state x_{Bβ}, which is a state of the cell of the second cell line after administering a second drug β, and a second-first perturbed state x_{Aβ}, which is a state of the cell of the first cell line (A) after administering the second drug, a first cell state x_{A}, which is a state of a cell of the first cell line, a first-second perturbed state x_{B}, which is a state of a cell of a second cell line B after administering the first drug α; and a second cell state x_{B}, which is a state of a cell of the second cell line; independently inputting, by the computing apparatus, the first-first perturbed state x_{Aα}, the first-second perturbed state x_{B}a, the second-second perturbed state x_{B}p, the first cell state x_{A}, and the second cell state x_{B} into the encoder to generate a first-first latent perturbed state z_{Aα}, a first-second latent perturbed state z_{Bα}, a second-second latent perturbed state z_{Bβ}, a first latent cell state z_{A}, a first-second latent perturbed state z_{Bα}, and a second latent cell state z_{B}, respectively; inputting, by the computing apparatus, a latent state z′_{Aβ}, which is obtained by adding the second-second latent perturbed state z_{Bβ} to a value obtained by subtracting the first-second latent perturbed state z_{Ba} from the first-first latent perturbed state z_{Aα}, into the generator to generate a second-first reconstructed perturbed state x′_{Aβ}; generating, by the computing apparatus, a first perturbation vector z_{Aa} by subtracting the first latent cell state z_{A} from the first-first latent perturbed state z^{A}_{α}; and generating a second perturbation vector z^{B}_{α} by subtracting the second latent cell state z_{B} from the first-second latent perturbed state z_{Bα}; and training, by the computing apparatus, the encoder using a loss L_{G}, which includes a first loss Lₜᵣᵢₚₗₑ, which includes a value obtained by subtracting the second-first perturbed state x_{Aβ} from the second-first reconstructed perturbed state x′_{Aβ}, and a second loss L_{delta}, which includes a value obtained by subtracting the second perturbation vector z^{B}_{α} from the first perturbation vector z^{A}_{α}. Here, the encoder training instruction codes may include a third set of instruction codes to execute the aforementioned steps.

Here, in the method for determining the suitability of an administered drug for cell state transition, the computing apparatus, and the non-volatile storage medium, the set of baseline states 101, the k-th set of perturbed states 102, the initial cell state 103, and the target cell state 104 may be array data consisting of expression levels of the genes in each corresponding cell, defined in a predetermined cell state space 100; the set of latent baseline states 201, the k-th set of latent perturbed states 202, the latent initial cell state 203, and the latent target cell state 204 are each values defined in a predetermined latent space 200; and the encoder used by the computing apparatus for encoding is configured to transform values belonging to the cell state space 100 into values belonging to the latent space 200.

Here, in the method for determining the suitability of an administered drug for cell state transition, the computing apparatus, and the non-volatile storage medium, the set of baseline states 101, the k-th set of perturbed states 102, the initial cell state 103, and the target cell state 104 may be array data consisting of expression levels of the genes in each corresponding cell, defined in a predetermined cell state space 100; the set of latent baseline states 201, the k-th set of latent perturbed states 202, the latent initial cell state 203, and the latent target cell state 204 may be each values defined in a predetermined latent space 200; and the encoder used by the computing apparatus for encoding may be configured to transform values belonging to the cell state space 100 into values belonging to the latent space 200. Also, the method for determining suitability may further comprise the steps of: acquiring, by the computing apparatus, a first state, which is a state of a predetermined cell; transforming, by the computing apparatus, the first state into the first latent state defined in the latent space using the encoder; determining, by the computing apparatus, a starting point represented by the first latent state in the latent space, determining, by the computing apparatus, an endpoint displaced by the k-th representative distance vector from the starting point, and determining, by the computing apparatus, multiple intermediate points on a straight line connecting the starting point and the endpoint; and inputting, by the trained generator, values of the starting point, the multiple intermediate points, and the endpoint to output reconstructed states belonging to the cell state space 100, corresponding to these values. Here, the reconstructed states may be the states along a transition path of states of the first cell when the k-th drug is administered.

Here, in the method for determining the suitability of an administered drug for cell state transition, the computing apparatus, and the non-volatile storage medium, the initial state of the specific cell line may be a disease state with a particular disease present in a specific cell line, and the target state of the specific cell line may be a normal state with the particular disease absent in the specific cell line.

Here, the particular disease may be cancer.

Here, the first subject may be a human.

Here, the first subject may be an animal or a plant other than a human.

According to another aspect of the present invention, a first method comprising the following steps may be provided. The first method may utilize the results of calculating representative distance vectors in a predetermined latent space for a plurality of different drugs. In other words, if a total of K drugs are given, a total of K representative distance vectors may be calculated, and the method may utilize these total K representative distance vectors.

Here, the method for calculating the k-th representative distance vector, which is a representative distance vector for k-th drug, may include the following steps (k=1, . . . , K). In other words, the computing apparatus may perform the steps of obtaining, by the computing apparatus, a set of baseline states 101, which is data representing a state of a set of actual cells, and after administering the k-th drug to the set of actual cells, obtain the k-th set of perturbed states 102, which is data representing the transitioned state of the set of actual cells; encoding, by the computing apparatus, the set of baseline states 101 and the k-th set of perturbed states 102 using a specific encoder to generate a set of latent baseline states 201 and the k-th set of latent perturbed states 202, respectively; calculating, by the computing apparatus, the k-th representative distance vector, which represents the distance between the set of latent baseline states and the k-th set of latent perturbed states.

Furthermore, the first method may include the following steps to determine the optimal drug for a specific cell line of a predefined first subject. These steps may include: obtaining, by the computing apparatus, the initial cell state 103, which is data representing the initial state of the specific cell line of the first subject, and the target cell state 104, which is data representing the target state of the specific cell line of the first subject; encoding, by the computing apparatus, the initial cell state 103 and the target cell state 104 using the encoder to generate the latent initial cell state 203 and the latent target cell state 204; calculating, by the computing apparatus, the reference distance vector (DR), which is the distance vector between the latent initial cell state and the latent target cell state; determining, by the computing apparatus, the representative distance vector among the total K representative distance vectors that is most similar to the reference distance vector (DR); and determining, by the computing apparatus, the drug corresponding to the most similar representative distance vector as the optimal drug for the specific cell line of the first subject.

### Advantageous Effects

According to the present invention, it is possible to provide a GAN model that can simulate perturbations through arithmetic operations within a latent space.

According to the present invention, it is also possible to provide a technology for finding optimal perturbations to transition the state of a cell represented within the latent space to a desired state.

### BRIEF DESCRIPTION OF DRAWINGS

FIG. 1 shows a configuration of a computing apparatus provided according to an embodiment of the present invention.

FIG. 2 shows a configuration of a computing apparatus provided according to an embodiment of the present invention.

FIG. 3 shows a process of encoding a set of baseline states observed from a set of actual cells into a set of latent baseline states belonging to a predetermined latent space according to an embodiment of the present invention.

FIG. 4 is a table showing states of N biomolecules observed from N selected actual cells.

FIG. 5 shows a concept of transforming values included in the cell state space into values included in the latent space.

FIG. 6 shows a process of encoding multiple baseline states observed from a set of actual cells into latent baseline states defined in the latent space according to an embodiment of the present invention.

FIG. 7 is a diagram showing the concept of administering multiple types of drugs according to an embodiment of the present invention.

FIG. 8 is a table explaining the change in the state of a cell when different drugs are administered.

FIG. 9 illustrates new states, k-th set of perturbed states, that N cells have when k-th drug is administered to N cells each having N baseline states included in the cell state space according to an embodiment of the present invention.

FIG. 10 shows a concept of encoding and transforming k-th set of perturbed states included in the cell state space into k-th set of latent perturbed states according to an embodiment of the present invention.

FIG. 11 is a flowchart showing the method of calculating the k-th representative distance vector, an indicator related to the administration of the k-th drug, according to an embodiment of the present invention.

FIG. 12 shows the notation of a set of latent baseline states and a k-th set of latent perturbed states constituting the present invention.

FIG. 13 is a conceptual diagram showing a transition phenomenon of a latent state observed as a result of the administration of the k-th drug according to an embodiment of the present invention.

FIG. 14 is a flowchart showing a method of generating the reference distance vector, which represents the difference between a predetermined initial cell state and a predetermined target cell state according to an embodiment of the present invention.

FIG. 15 shows a concept of generating a latent initial cell state and a latent target cell state by encoding an initial cell state and a target cell state according to an embodiment of the present invention.

FIG. 16 shows the method of calculating the suitability related to the administration of the k-th drug according to an embodiment of the present invention.

FIG. 17 is a diagram showing the concept of calculating the degree of similarity between different representative distance vectors calculated according to the administration of different drugs and the reference distance vector.

FIG. 18 is a table showing the concept of calculating the degree of similarity between different representative distance vectors calculated according to the administration of different drugs and the reference distance vector.

FIG. 19 shows a structure of an encoder training unit according to an embodiment of the present invention, which trains the encoder presented in FIG. 1, FIG. 3, FIG. 6, FIG. 10, FIG. 14, and FIG. 15.

FIG. 20A is a flowchart showing a method of calculating the loss used to train the encoder and training the encoder using the loss according to an embodiment of the present invention.

FIG. 20B is a diagram expressing the method shown in FIG. 20A.

FIG. 21A is a flowchart showing a method of calculating the loss used to train the encoder and training the encoder using the loss according to another embodiment of the present invention.

FIG. 21B is a diagram expressing the method shown in FIG. 21A.

FIG. 22 compares the results of training the encoder using the first and/or second loss defined according to the embodiments of the present invention explained in FIG. 20A to FIG. 21B with the results of training the encoder using a differently defined loss.

FIG. 23A shows a concept of vector α′k (=D[k]) representing the effect of k-th drug (αₖ) defined in the latent space and dividing the vector D[k] by p and the p+1 points defined in the latent space obtained thereby.

FIG. 23B shows a concept of obtaining p+1 reconstructed cell states defined in the cell state space by inputting the p+1 points into the trained generator explained through FIG. 19 to FIG. 21B, respectively, according to the k-th drug (αₖ).

FIG. 24 is a diagram explaining the meaning of the k-th representative distance vector corresponding to the administration of the k-th drug explained in FIG. 13.

### BEST MODE FOR PRACTICING THE INVENTION

Hereinafter, embodiments of the present invention will be described with reference to the attached drawings. However, the present invention is not limited to the embodiments described in this disclosure and may be implemented in various other forms. The terms used in this disclosure are for aiding the understanding of the embodiments and are not intended to limit the scope of the present invention. Also, singular forms used herein include plural forms unless the context clearly indicates otherwise.

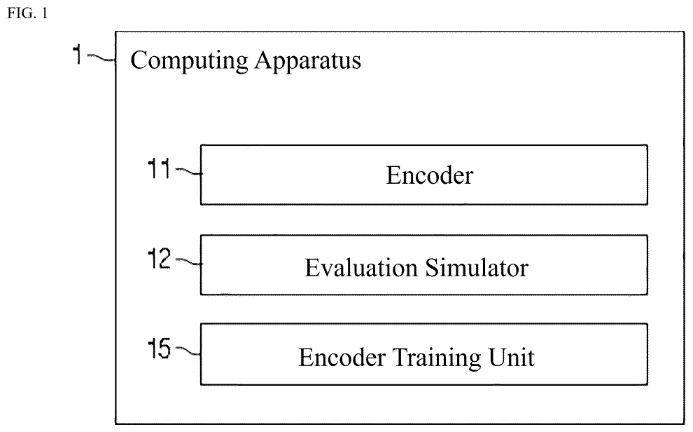

FIG. 1 shows a configuration of a computing apparatus provided according to an embodiment of the present invention.

The computing apparatus 1 may include an encoder 11, an evaluation simulator 12, and an encoder training unit 15. The functions of the encoder 11, the evaluation simulator 12, and the encoder training unit will be described later.

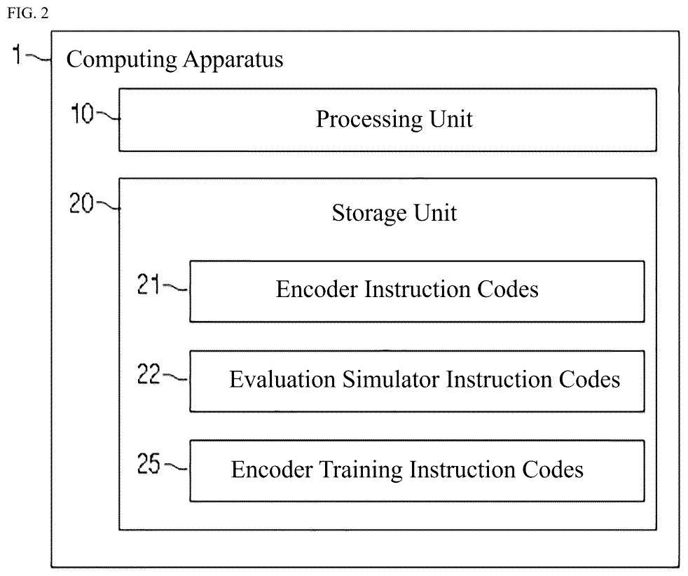

FIG. 2 shows a configuration of a computing apparatus provided according to an embodiment of the present invention.

The computing apparatus 1 may include a processing unit 10 and a storage unit 20. The storage unit 20 may include encoder instruction codes 21, evaluation simulator instruction codes 22, and encoder training instruction codes 25. The encoder 11 in FIG. 1 may be implemented by the processing unit 10 which reads and executes the encoder instruction codes 21, the evaluation simulator 12 may be implemented by the processing unit 10 which reads and executes the evaluation simulator instruction codes 22, and the encoder training unit 15 may be implemented by the processing unit 10 which reads and executes the encoder training instruction codes 25.

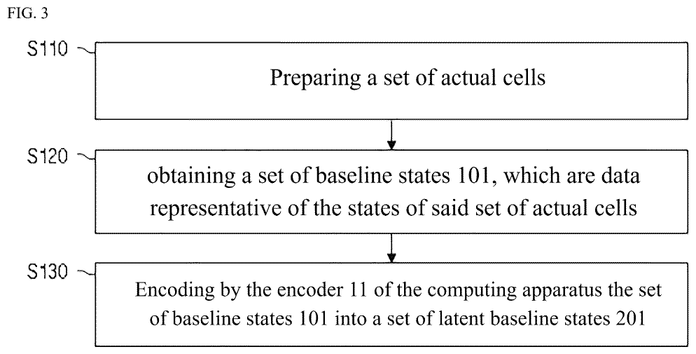

FIG. 3 shows a process of encoding a set of baseline states observed from a set of actual cells into a set of latent baseline states belonging to a predetermined latent space according to an embodiment of the present invention.

In step S110, a set of actual cells can be prepared. The set of actual cells may include disease cells and/or normal cells obtained from a single tissue. Also, the set of actual cells may be obtained from patients and/or non-patients.

The single tissue may be a single tissue selected from, for example, lung, liver, stomach, brain, etc.

In an embodiment of the present invention, the disease cells may be cancer cells or cells experiencing a disease other than cancer.

In step S120, a set of baseline states 101, which are data representing the states of the set of actual cells, may be acquired.

The combination of genes predominantly expressed in the set of actual cells obtained from a given single tissue may differ from the combination of genes predominantly expressed in cells obtained from another tissue. For example, the combination of genes predominantly expressed in stomach cells may differ from the combination of genes predominantly expressed in liver cells.

The data representing the states of the set of actual cells may be an array consisting of the expression levels of the genes predominantly expressed in the set of actual cells. That is, if the total number of genes predominantly expressed in the set of actual cells is M, the state of each actual cell may be an array consisting of M numerical values. Since the expression levels of genes may vary slightly in different cells, the states of different cells may have slightly different values.

In one embodiment, the expression levels of genes expressed in each cell may be related to the molecular weight of biomolecules, such as proteins, produced by the genes.

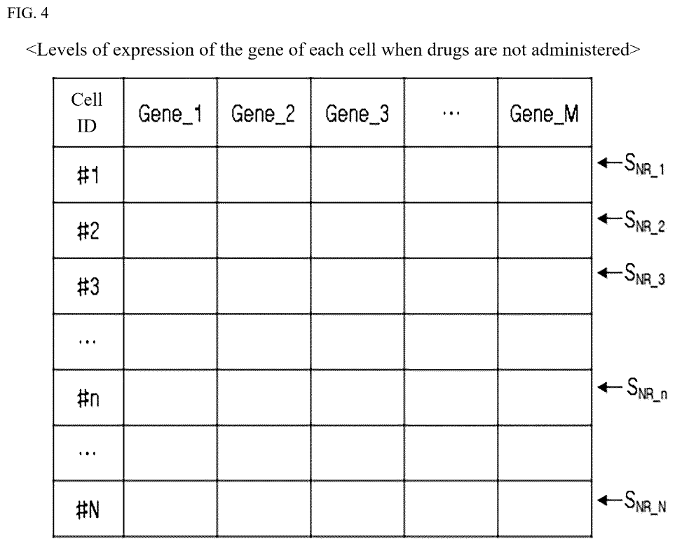

FIG. 4 is a table showing states of N biomolecules observed from N selected actual cells.

In the table of FIG. 4, the “Cell ID” field is a value that identifies the different N cells. The N cells can all be homologous cells obtained from a single tissue. For example, all of the N cells may be liver cells or neurons.

The table in FIG. 4 has M genes expressed in the N cells as fields. At the intersection of each row and each column in the table, the amount of expression of the gene indicated by the column in the individual cell indicated by the row can be recorded.

That is, in the table of FIG. 4, the state of cell #n can be defined as a “gene expression level vector” consisting of M values constituting the row designated by #n.

In this disclosure, the set of all possible vectors as the “gene expression level vector” can be defined as the cell state space 100.

For example, the baseline states observed from the N cells in step S120 can be the N gene expression level vectors found in the N rows presented in FIG. 4. In FIG. 4, the N baseline states are presented as reference numerals SNR_1, SNR_2, SNR_3, . . . SNR_n, . . . and SNR_N.

In step S130, the encoder 11 of the computing apparatus may encode the set of baseline states 101 into a set of latent baseline states 201.

Step S130 can be considered a step of transforming the N baseline states included in the cell state space into vectors of a different space. In this disclosure, the different space can be referred to as the “latent space.”

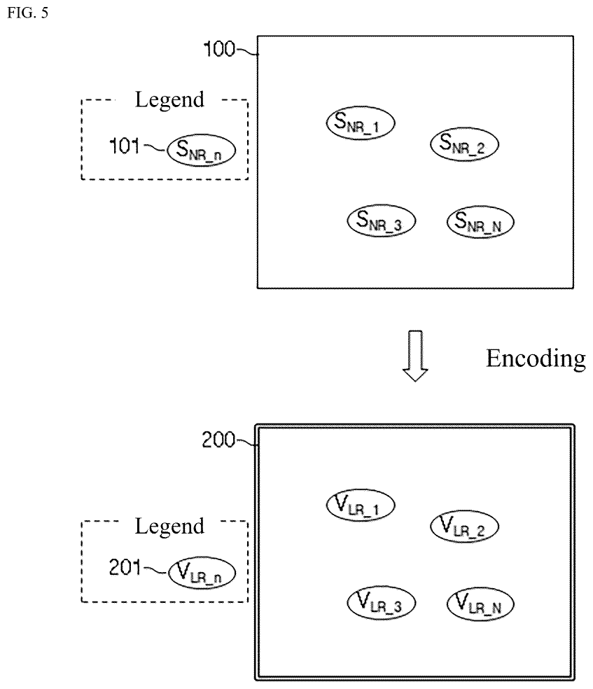

FIG. 5 shows a concept of transforming values included in the cell state space 100 into values included in the latent space 200 as presented in step S130.

For example, the encoder 11 transforms N baseline states SNR_1, SNR_2, SNR_3, . . . SNR_n, . . . and SNR_N included in the cell state space 100 into N latent baseline states VLR_1, VLR_2, VLR_3, . . . VLR_n, . . . and VLR_N included in the latent space 200. In this disclosure, the process of transforming each of the N baseline states 101 into the N latent baseline states 201 by the encoder 11 can be referred to as “encoding” or “encoding process.”

An example of the process of preparing the encoder 11 will be described later.

Each of the latent baseline states 201 can be a vector.

When each baseline state 101 is an M-dimensional vector, each latent baseline state 201 may or may not be an M-dimensional vector.

In this disclosure, the set of all possible vector values that latent baseline state 201 can be defined as the latent space 200.

A set of latent baseline states 201 generated by the method presented in FIG. 3 can be used for the calculation of perturbation scores described later.

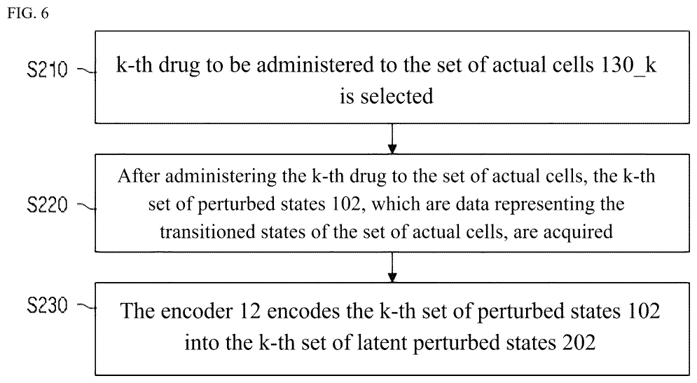

FIG. 6 shows a process of encoding a set of baseline states observed from a set of cells into states in the latent space according to an embodiment of the present invention.

In step S210, the k-th drug to be administered to the set of actual cells 130_k among the K drugs can be selected.

The subject of the selection can be a computing apparatus or a human.

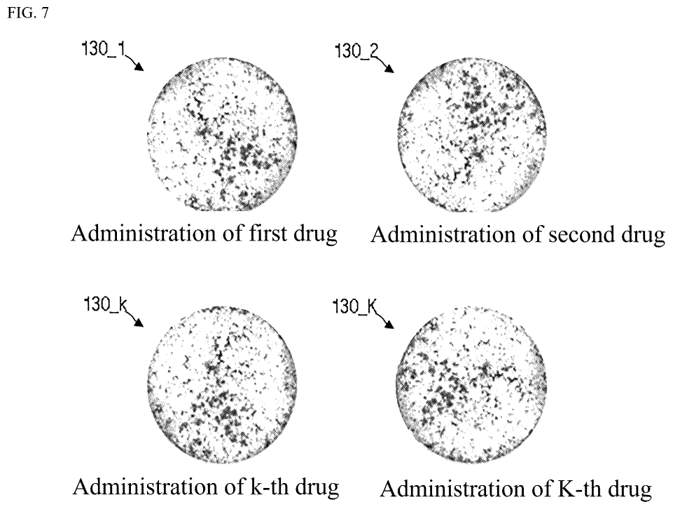

FIG. 7 shows a concept of administering K drugs to a set of cells presented in step S210.

The k-th drug may be a drug that inhibits the expression of a specific gene among the genes expressed in the actual cells. Different drugs can inhibit the expression of different genes.

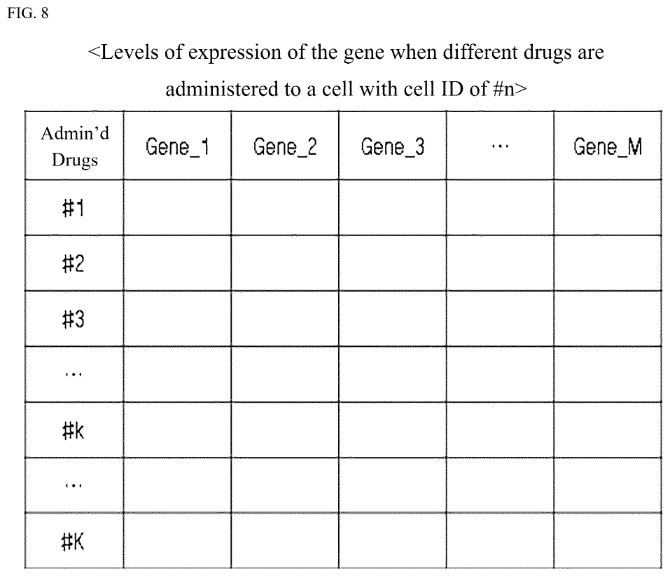

FIG. 8 is a table explaining the change in the state of a cell when different drugs are administered.

Each row in the table presented in FIG. 8 represents a different drug, and each column represents the expression levels of M genes expressed in a given specific cell. The K different cases of perturbing a cell with cell ID #n by administering K different drugs can be considered. Once a drug is administered to a cell, it is impossible to perfectly revert the state of the cell to its original state, and finding two cells with exactly the same state is practically difficult, so the experiment of administering K different drugs to the cell with cell ID #n is practically impossible, but here it is presented as a thought experiment to aid the understanding of the present invention. The combination of expression levels of M genes in a new state (attractor) to which the cell transitions as a result of the perturbation of the cell with each drug may differ for each drug. The administration of one selected drug among the several possible drugs presented in FIG. 7 can correspond to one of the rows in the table presented in FIG. 8.

Returning to FIG. 6, in step S220, after administering the k-th drug to the set of actual cells, the k-th set of perturbed states 102, which are data representing the transitioned states of the set of actual cells, may be acquired.

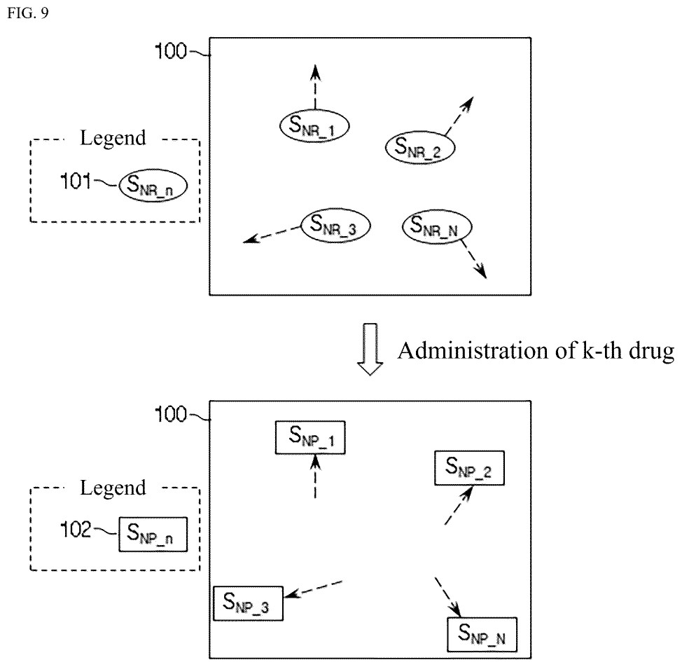

FIG. 9 illustrates the k-th set of perturbed states 102, which are new states acquired by administering the k-th drug to N cells each having N baseline states included in the cell state space 100 according to an embodiment of the present invention. The k-th set of perturbed states 102 consists of a total of N perturbed states.

In the example shown in FIG. 9, the baseline states S_{NR_1}, S_{NR_2}, S_{NR_3}, . . . SN_{R_n}, . . . and SN_{R_N} are each transitioned to perturbed states S_{NP_1}, S_{NP_2}, S_{NP_3}, . . . SN_{P_n}, . . . and SN_{P_N}, respectively.

Returning to FIG. 6, in step S230, the encoder 11 may encode the k-th set of perturbed states 102 into the k-th set of latent perturbed states 202.

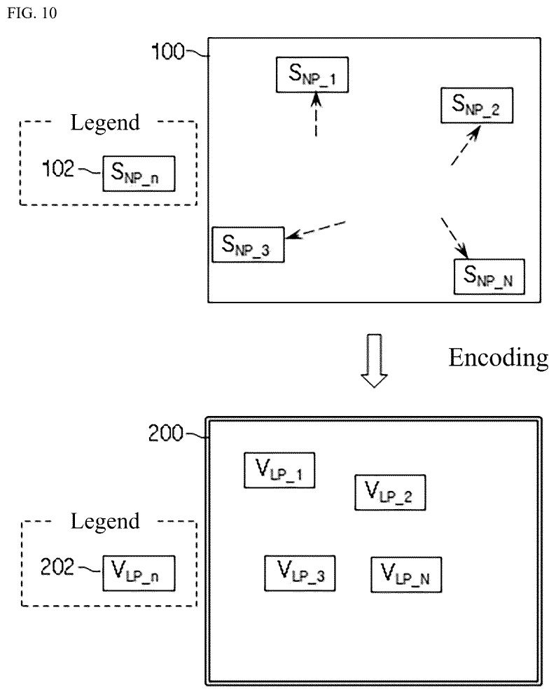

FIG. 10 shows a concept of the k-th set of latent perturbed states 202, which are transformed by encoding the k-th set of perturbed states 102 included in the cell state space 100 according to an embodiment of the present invention. The k-th set of latent perturbed states 202 consists of a total of N latent perturbed states and belongs to the latent space 200.

In the example shown in FIG. 10, the perturbed states S_{NP_1}, S_{NP_2}, S_{NP_3}, . . . SN_{P_n}, . . . and SN_{P_N} are each transformed to latent perturbed states V_{LP_1}, V_{LP_2}, V_{LP_3}, . . . V_{LP_n}, . . . and V_{LP_N}, respectively.

The k-th set of latent perturbed states 202 generated by the method presented in FIG. 6 can be used for the calculation of perturbation scores, which will be described later, along with the set of latent baseline states 201 presented in FIG. 3.

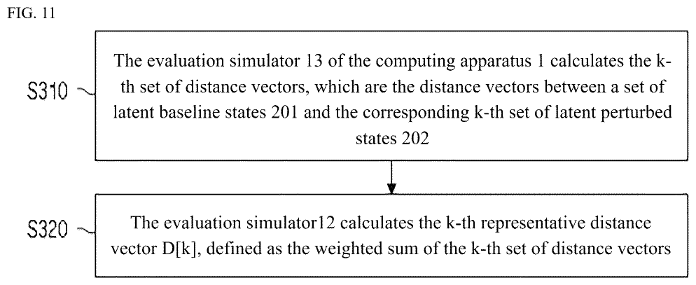

FIG. 11 is a flowchart showing a method of calculating the k-th representative distance vector, an indicator related to the administration of the k-th drug, according to an embodiment of the present invention.

In step S310, the evaluation simulator 12 of the computing apparatus 1 can calculate the k-th set of distance vectors, which are the distance vectors between a set of latent baseline states 201 and the corresponding k-th set of latent perturbed states 202.

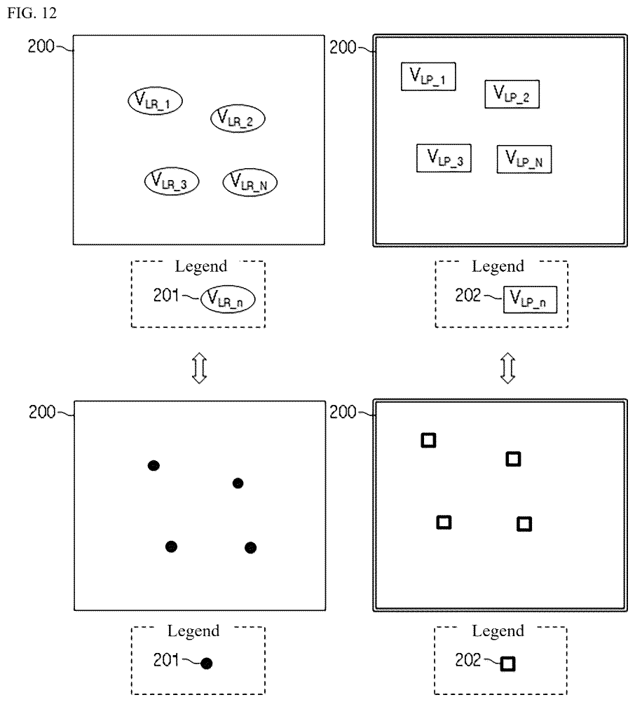

FIG. 12 shows the notation of a set of latent baseline states 201 and the k-th set of latent perturbed states 202 constituting the present invention.

For the convenience of explanation, as shown in FIG. 12, each latent baseline state 201 may be expressed as a dot and each latent perturbed state 202 may be expressed as a square. The set of latent baseline states 201 in FIG. 12 is presented in FIG. 5, and the k-th set of latent perturbed states 202 is presented in FIG. 10.

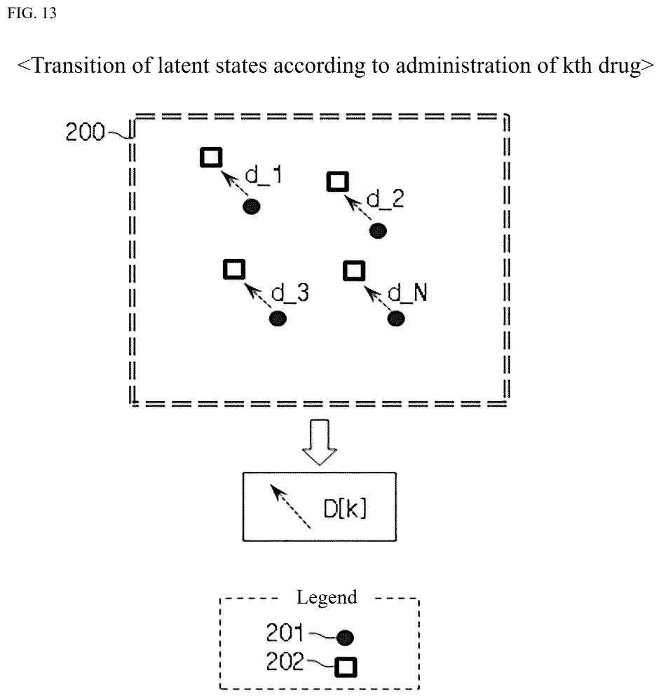

FIG. 13 is a conceptual diagram showing a transition phenomenon of a latent state observed as a result of administering the k-th drug to a cell according to an embodiment of the present invention.

Since the set of latent baseline states 201 and the k-th set of latent perturbed states 202 presented in FIG. 12 both belong to the latent space 200, they can be expressed together in the latent space 200 as shown in FIG. 13. The arrows presented in FIG. 13 indicate the vectors representing the difference between each latent baseline state 201 and the corresponding latent perturbed state 202 within the latent space 200. For example, reference numeral d_3 represents the distance between the third latent baseline state and the corresponding third latent perturbed state.

As shown in FIG. 13, the present invention can define the set of distance vectors between a set of latent baseline states 201 and the corresponding k-th set of latent perturbed states 202 as the k-th set of distance vectors.

In step S320, the evaluation simulator 12 can calculate the k-th representative distance vector D[k], defined as the weighted sum of the k-th set of distance vectors.

In the example shown in FIG. 13, the k-th representative distance vector D[k] can be calculated as shown in Equation 1.

D[k]=Σ_{from n=1}^{to n=N}(d_n*w_n [Equation 1]
where w_n is the weight defined corresponding to d_n.

The k-th representative distance vector D[k] generated by the method presented in FIG. 11 can be used for the calculation of perturbation scores described later.

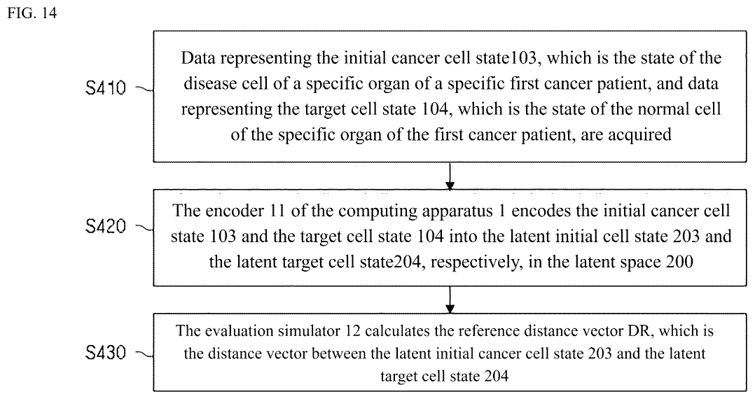

FIG. 14 is a flowchart showing a method of generating the reference distance vector DR representing the difference between a predetermined initial cell state and a predetermined target cell state according to an embodiment of the present invention.

In an embodiment of the present invention, the initial cell state can be the state of a disease cell deviating from a healthy state. The target cell state can be the state of a healthy cell. Here, the cell having the initial cell state and the cell having the target cell state can be cells of the same single tissue. For example, both the cell having the initial cell state and the cell having the target cell state can be liver cells.

In an embodiment of the present invention, the disease cell can be a cancer cell. That is, the initial cell state can be the state of a cancer cell. However, the present invention is not limited thereto, and the disease cell can be a cell suffering from a disease other than cancer.

In another embodiment of the present invention, the initial cell state can be the state of a healthy cell, and the target cell state can be the state of an unhealthy cell. In such an embodiment, the present invention can be utilized for purposes other than therapeutic purposes for a patient, such as other research purposes.

In step S410, data representing the initial cell state 103, which is the state of the disease cell of a specific organ of a specific first patient, and data representing the target cell state 104, which is the state of the normal cell of the specific organ of the first patient, may be acquired.

In one embodiment, the first patient can be the first cancer patient.

For example, the initial cell state 103 can be a gene expression level vector consisting of the expression levels of M genes observed in the disease cell of a specific organ of the first patient. A target cell state 104 can be a gene expression level vector consisting of the expression levels of M genes observed in the normal cell of the specific organ of the first patient.

In step S420, the encoder 11 of the computing apparatus 1 may encode the initial cell state 103 and the target cell state 104 into the latent initial cell state 203 and the latent target cell state 204, respectively, in the latent space 200.

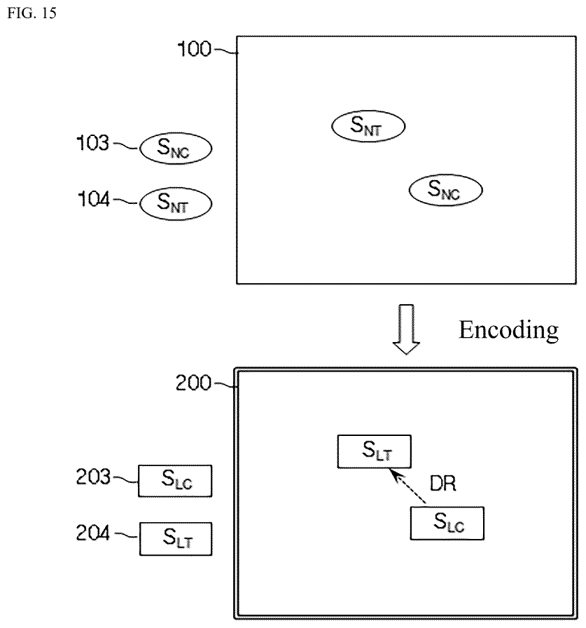

FIG. 15 shows a concept of generating the latent initial cell state 203 and the latent target cell state 204 by encoding the initial cell state 103 and the target cell state 104.

In FIG. 15, the initial cell state 103 and the target cell state 104 are denoted by reference numerals S_{NC} and S_{NT}, respectively, and the latent initial cell state 203 and the latent target cell state 204 are denoted by reference numerals S_{LC} and S_{LT}, respectively. The initial cell state 103 and the target cell state 104 are included in the cell state space 100, and the latent initial cell state 203 and the latent target cell state 204 are included in the latent space 200.

In step S430, the evaluation simulator 12 of the computing apparatus 1 can calculate the reference distance vector DR, which is the distance vector between the latent initial cell state 203 and the latent target cell state 204.

The arrow DR shown in FIG. 15 indicates the vector representing the difference between the latent initial cell state 203 and the latent target cell state 204 within the latent space 200, which means the reference distance vector DR of step S430.

The reference distance vector DR generated by the method presented in FIG. 14 can be used for the calculation of perturbation scores described later.

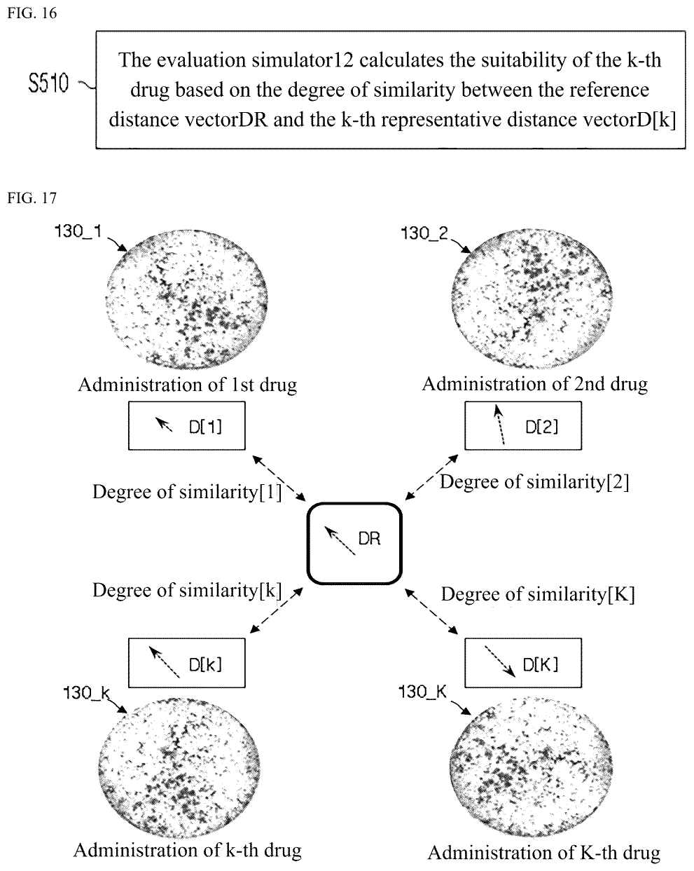

FIG. 16 shows the method of calculating the suitability of the administration of the k-th drug provided according to an embodiment of the present invention.

In step S510, the evaluation simulator 12 of the computing apparatus 1 can calculate the suitability of the k-th drug based on the degree of similarity between the reference distance vector DR and the k-th representative distance vector D[k]. The suitability of the k-th drug can be referred to as the perturbation score of the k-th drug.

In a preferred embodiment, the higher the degree of similarity between the reference distance vector DR and the k-th representative distance vector D[k], the greater the value of the suitability of the k-th drug.

FIG. 17 is a diagram showing the concept of calculating the degree of similarity between different representative distance vectors calculated according to the administration of different drugs and the reference distance vector DR.

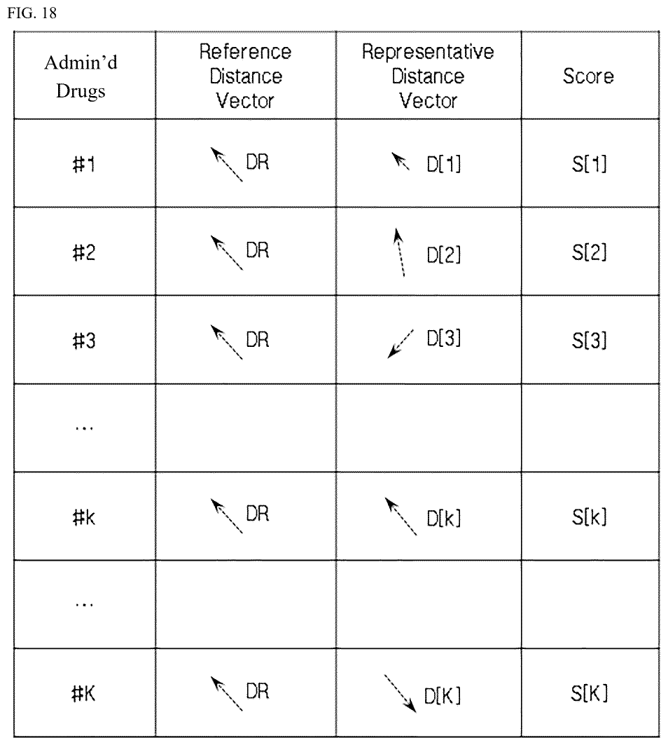

FIG. 18 is a table showing the concept of calculating the degree of similarity between different representative distance vectors calculated according to the administration of different drugs and the reference distance vector DR.

FIGS. 3 to 13 illustrate a process of calculating the k-th representative distance vector D[k] for the selected k-th drug among multiple drugs. Applying the series of processes presented in FIGS. 3 to 13 to each of the total K drugs, the first representative distance vector D[1] to the K-th representative distance vector D[K] can be calculated.

Different representative distance vectors calculated for different drugs can have different values.

In FIGS. 17 and 18, the different representative distance vectors D[1], D[2], . . . D[k], . . . D[K] calculated according to the administration of the first drug, second drug, k-th drug, and K-th drug are illustrated as arrows. The direction and magnitude of the arrows representing D[1], D[2], . . . D[k], . . . D[K] are expressed differently, indicating that D[1], D[2], . . . D[k], . . . D[K] have different values.

It can be understood that the degree of similarity[1] between the reference distance vector DR and D[1], the degree of similarity[2] between the reference distance vector DR and D[2], the degree of similarity[k] between the reference distance vector DR and D[k], and the degree of similarity[K] between the reference distance vector DR and D[K] can be calculated.

In the example shown in FIGS. 17 and 18, since D[k] is the most similar to the reference distance vector DR, it can be determined that the third drug among the total K drugs has the highest suitability (perturbation score S). Therefore, it can be determined that the third drug is the most suitable drug among the total K drugs. Here, the drug may be a combination of two or more drugs.

In FIGS. 14 and 15, reference numerals 103 and 104 are examples of the initial cell state and the target cell state (the state of a normal cell), respectively, presented for the purpose of transforming the disease cell into a normal cell. However, the present invention is not limited thereto, and reference numerals 103 and 104 can be defined as any state selected for a specific purpose. The specific purpose may include therapeutic purposes and other research purposes.

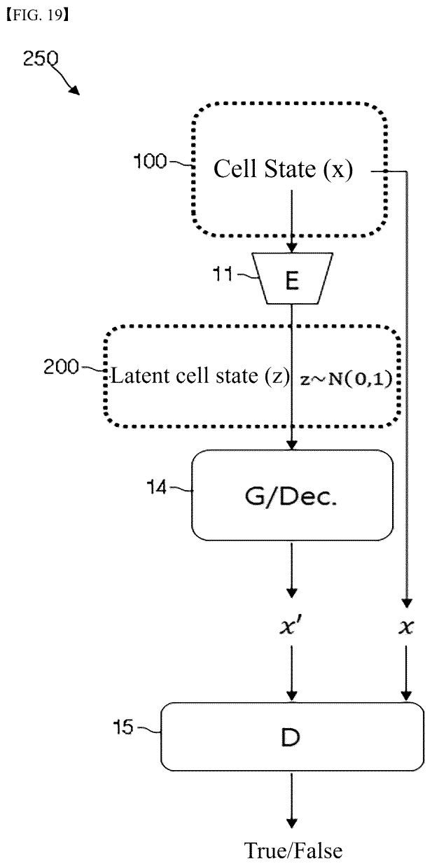

FIG. 19 shows a structure of an encoder training unit 15 for training the encoder presented in FIGS. 1, 3, 6, 10, 14, and 15 according to an embodiment of the present invention.

The encoder training unit 15 may be implemented by the processing unit 10 which reads and executes the encoder training instruction codes 25 stored in the storage unit 20.

The encoder 11 can be configured to include a neural network.

The encoder 11 can be trained using a well-known VAE (Variational AutoEncoder) and GAN (Generative Adversarial Network) technologies.

In FIG. 19, reference numerals 11 and 14 are the encoder and the decoder of VAE, respectively. Reference numerals 14 and 15 are the generator and the discriminator of GAN, respectively. That is, reference numeral 14 serves as both the decoder of VAE and the generator of GAN.

The data input to the encoder 11 are data included in the cell state space 100 described above. That is, the cell state x observed in the actual cell can be input to the encoder 11. The cell state can be, for example, the gene expression level data SNR_n presented in FIG. 4. The cell state x is input to discriminator 15.

The values output by the encoder 11 constitute the latent space 200 described above. The encoder 11 can output the latent cell state z, which is the value obtained by transforming the cell state x. The latent cell state z can be a vector, and each element of the vector can follow a normal probability distribution with μ=0 and σ²=1.

The latent cell state z is input to the generator/decoder 14. The generator/decoder 14 generates and outputs the reconstructed cell state x′ from the latent cell state z. The reconstructed cell state x′ is input to discriminator 15.

The discriminator 15 determines whether the reconstructed cell state x′ is the same as the cell state x observed in the actual cell.

The encoder training unit 15 can define a predetermined loss and train the encoder 11, generator/decoder 14, and discriminator 15 to reduce the loss.

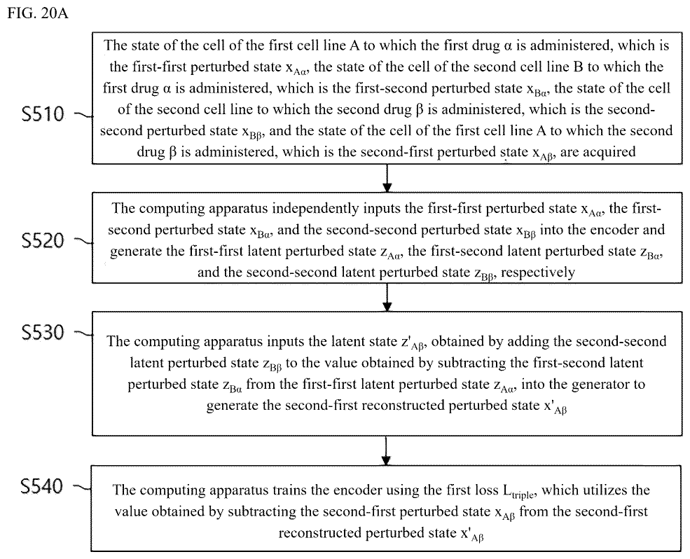

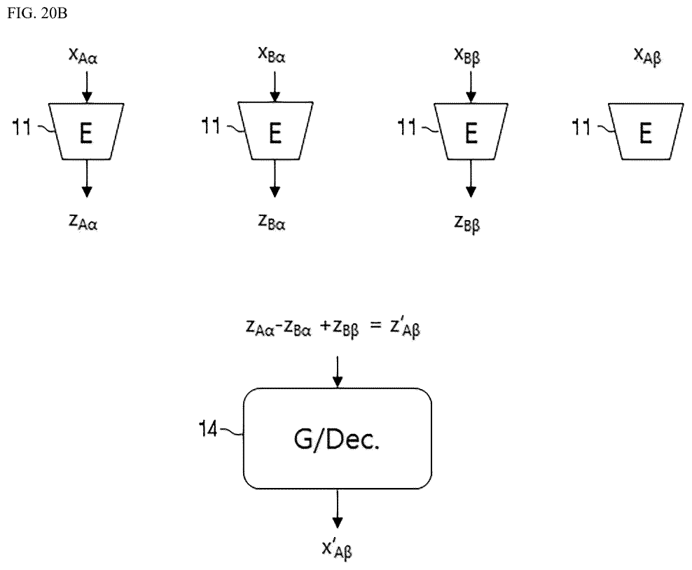

FIG. 20A is a flowchart showing a method of calculating the loss used to train the encoder and training the encoder using the loss according to an embodiment of the present invention.

FIG. 20B is a diagram expressing the method shown in FIG. 20A.

Hereinafter, FIGS. 20A and 20B will be described together.

In step S510, the state of the cell of the first cell line A to which the first drug α is administered, which is the first-first perturbed state x_{Aα}, the state of the cell of the second cell line B to which the first drug α is administered, which is the first-second perturbed state x_{Bα}, the state of the cell of the second cell line to which the second drug β is administered, which is the second-second perturbed state x_{Bβ}, and the state of the cell of the first cell line A to which the second drug β is administered, which is the second-first perturbed state x_{Aβ}, may be acquired.

This acquisition can be performed using RNA sequencing, for example. The information obtained using RNA sequencing may be acquired by the computing apparatus in the form of data.

In one example, the first cell line and the second cell line can both be obtained from a specific single body tissue (e.g., liver). The first cell line may be obtained from the specific single body tissue of the first person (e.g., the liver of the first person), and the second cell line may be obtained from the specific single body tissue of the second person (e.g., the liver of the second person). The first cell line may have a disease cell state or a normal cell state without disease. The second cell line may have a disease cell state or a normal cell state without disease.

In step S520, the computing apparatus can independently input the first-first perturbed state x_{Aα}, the first-second perturbed state x_{Bα}, and the second-second perturbed state x_{Bβ} into the encoder and generate the first-first latent perturbed state z_{Aα}, the first-second latent perturbed state z_{Bα}, and the second-second latent perturbed state z_{Bβ}, respectively.

That is, the first-first perturbed state x_{Aa} can be input to the encoder to generate the first-first latent perturbed state z_{Aα}. The first-second perturbed state x_{Ba} can be input to the encoder to generate the first-second latent perturbed state z_{Bα}. The second-second perturbed state x_{Bβ} can be input to the encoder to generate the second-second latent perturbed state z_{Bβ}.

In step S530, the computing apparatus can input the latent state z′_{Aβ}, obtained by adding the second-second latent perturbed state z_{Bβ} to the value obtained by subtracting the first-second latent perturbed state z_{Ba} from the first-first latent perturbed state z_{Aα}, into the generator to generate the second-first reconstructed perturbed state x′_{Aβ}.

In step S540, the computing apparatus can train the encoder using the first loss Lₜᵣᵢₚₗₑ, which utilizes the value obtained by subtracting the second-first perturbed state x_{Aβ} from the second-first reconstructed perturbed state x′_{Aβ}.

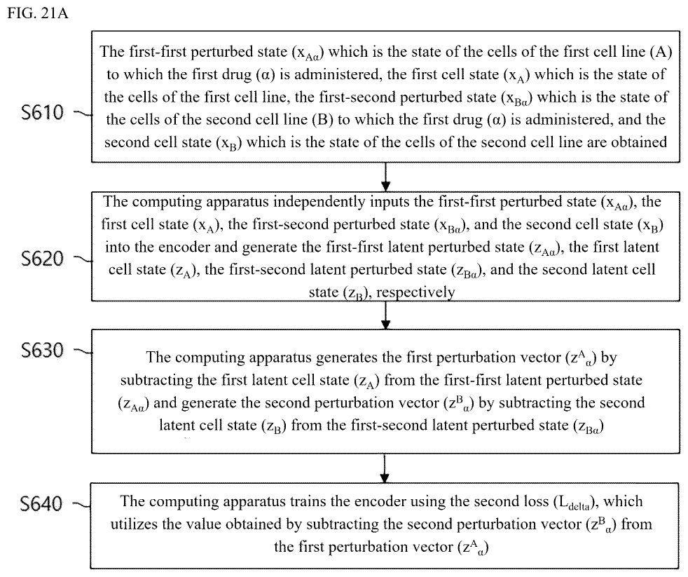

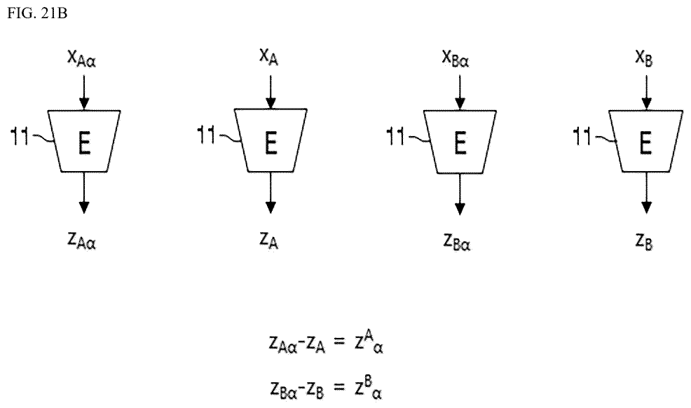

FIG. 21A is a flowchart showing a method of calculating the loss used to train the encoder and training the encoder using the loss according to another embodiment of the present invention.

FIG. 21B is a diagram expressing the method shown in FIG. 21A.

Hereinafter, FIGS. 21A and 21B will be described together.

In step S610, the following states of the cells can be obtained: the first-first perturbed state (x_{Aα}) which is the state of the cells of the first cell line (A) to which the first drug (α) is administered, the first cell state (x_{A}) which is the state of the cells of the first cell line, the first-second perturbed state (x_{Bα}) which is the state of the cells of the second cell line (B) to which the first drug (α) is administered, and the second cell state (x_{B}) which is the state of the cells of the second cell line.

This acquisition can be performed using RNA sequencing, for example.

In step S620, the computing apparatus can independently input the first-first perturbed state (x_{Aα}), the first cell state (x_{A}), the first-second perturbed state (x_{Bα}), and the second cell state (x_{B}) into the encoder and generate the first-first latent perturbed state (z_{Aα}), the first latent cell state (z_{A}), the first-second latent perturbed state (z_{Bα}), and the second latent cell state (z_{B}), respectively.

In step S630, the computing apparatus can generate the first perturbation vector (z^{A}_{α}) by subtracting the first latent cell state (z_{A}) from the first-first latent perturbed state (z_{Aα}) and generate the second perturbation vector (z^{B}_{α}) by subtracting the second latent cell state (z_{B}) from the first-second latent perturbed state (z_{Bα}).

In step S640, the computing apparatus can train the encoder using the second loss (L_{delta}), which utilizes the value obtained by subtracting the second perturbation vector (z^{B}_{α}) from the first perturbation vector (z^{A}_{α}).

According to another embodiment of the present invention, the method for training the encoder can use both the first loss (Lₜᵣᵢₚₗₑ) and the second loss (L_{delta}). This method can include the following steps.

In step (S710), the states of the cells can be obtained as follows: the first-first perturbation state (x_{Aα}), which is the state of the cell of the first cell line (A) to which the first drug (α) is administered; the first-second perturbation state (x_{Bα}), which is the state of the cell of the second cell line (B) to which the first drug (α) is administered; the second-second perturbation state (x_{Bβ}), which is the state of the cell of the second cell line to which the second drug (β) is administered; the second-first perturbation state (x_{Aβ}), which is the state of the cell of the first cell line (A) to which of second drug (β) is administered; the first cell state (x_{A}) which is the state of the cell of the first cell line; the first-second perturbed state (x_{Bα}), which is the state of the cell of the second cell line (B) to which the first drug (α) is administered; and the second cell state (x_{B}) which is the state of the cell of the second cell line.

In step S720, the computing apparatus can independently input the first-first perturbed state (x_{Aα}), the first-second perturbed state (x_{Bα}), the second-second perturbed state (x_{Bβ}), the first cell state (x_{A}), the first-second perturbed state (x_{Bα}), and the second cell state (x_{B}) into the encoder and generate the first-first latent perturbed state (z_{Aα}), the first-second latent perturbed state (z_{Bα}), the second-second latent perturbed state (z_{Bβ}), the first latent cell state (z_{A}), the first-second latent perturbed state (z_{Bα}), and the second latent cell state (z_{B}), respectively.

In step S730, the computing apparatus can input the latent state (z′_{Aβ}), obtained by adding the second-second latent perturbed state (z_{Bβ}) to the value obtained by subtracting the first-second latent perturbed state (z_{Bα}) from the first-first latent perturbed state (z_{Aα}), into the generator to generate the second-first reconstructed perturbed state (x′_{Aβ}). The computing apparatus can generate the first perturbation vector (z^{A}_{α}) by subtracting the first latent cell state (z_{A}) from the first-first latent perturbed state (z_{Aα}), and generate the second perturbation vector (z^{B}_{α}) by subtracting the second latent cell state (z_{B}) from the first-second latent perturbed state (z_{Bα}).

In step S740, the computing apparatus can train the encoder using the loss (L_{G}), which includes the sum of the first loss (Lₜᵣᵢₚₗₑ), which contains the value obtained by subtracting the second-first perturbed state (x_{Aβ}) from the second-first reconstructed perturbed state (x′_{Aβ}), and the second loss (L_{delta}), which contains the value obtained by subtracting the second perturbation vector (z^{B}_{α}) from the first perturbation vector (z^{A}_{α}).

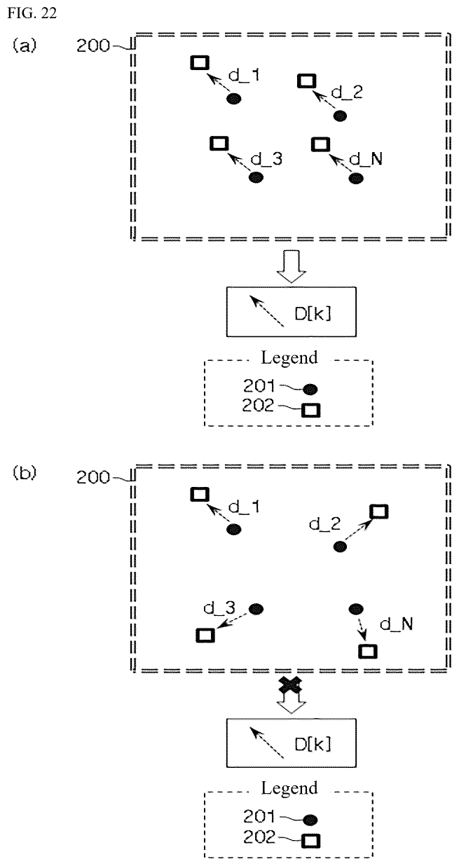

FIG. 22 compares the results of training the encoder using the first and/or second loss defined according to the embodiments of the present invention explained in FIGS. 20A to 21B with the results of training the encoder using a loss which is defined in a different manner.

FIG. 22 in (a) shows the results of training the encoder using the first and/or second loss defined according to the embodiments of the present invention explained in FIGS. 20A to 21B.

FIG. 22 in (a) presents a set of latent baseline states 201 and the k-th set of latent perturbed states 202, which have transitioned due to the administration of the k-th drug, within the latent space 200. The direction in which the set of latent baseline states 201 has moved to the k-th set of latent perturbed states 202 shows a certain tendency, indicating that the first and/or second loss was used in the process of training the encoder 11.

FIG. 22 in (b) shows the results of training the encoder using an arbitrary loss other than the first and/or second loss defined according to the embodiments of the present invention explained in FIGS. 20A to 21B.

FIG. 22 in (b) presents a set of latent baseline states 201 and the k-th set of latent perturbed states 202, which have transitioned due to the administration of the k-th drug, within the latent space 200. The direction in which the set of latent baseline states 201 has moved to the k-th set of latent perturbed states 202 shows no tendency, indicating that a different kind of loss was used in the process of training the encoder 11.

As described above, in the present invention, the set of distance vectors between the set of latent baseline states 201 and the corresponding k-th set of latent perturbed states 202 is defined as the k-th set of distance vectors, and the k-th representative distance vector D[k], defined as the weighted sum of the k-th set of distance vectors, is calculated. This is based on the assumption that the k-th set of distance vectors has a certain common tendency. However, as shown in FIG. 22 in (b), if there is no tendency or the tendency is weak among the k-th set of distance vectors, there is a problem that the calculated k-th representative distance vector D[k] may not represent the characteristics of the k-th drug.

In other words, the introduction of the first and/or second loss in the present invention is proposed to ensure that the output value of the encoder 11 trained based on these losses reveals the tendency of the effects of a specific drug that can be administered to cells.

The present invention can be used for drug repositioning purposes. The present invention can be used for drug repositioning purposes. For example, the k-th drug (αₖ), developed for the treatment of the first disease, can be simulated using the present invention to determine if it is effective for the treatment of the second disease, thereby discovering new uses for the k-th drug (αₖ).

The present invention can be used to simulate changes in gene expression levels of cells in response to perturbations (such as drugs, shRNA, etc.), to elucidate the intracellular mechanisms of these perturbations. For example, the change in gene expression levels in a given cell A in response to the k-th drug (αₖ) can be simulated in the latent space 100 as follows. The position of a given cell in the latent space can be defined as z_{A}, and the vector representing the effect of the k-th drug (am) can be defined as α′ₖ (=D[k]). Here, the vector representing the effect of the k-th drug (αₖ), α′ₖ, is the k-th representative distance vector D[k] shown in FIG. 13 (i.e., α′ₖ=D[k]).

By dividing the vector α′ₖ (=D[k]) into p equal parts, p+1 points (positions) in the latent space 100 may be obtained which are referred to as {z_{A}, z_{A}+(α′ₖ/p), z_{A}+2((α′ₖ/p), . . . z_{A}+p(α′ₖ/p)}={z_{A}+0*(D[k]/p), z_{A}+1*(D[k]/p), z_{A}+2*(D[k]/p), . . . z_{A}+p*(D[k]/p)}={D[k][0], D[k][1], D[k][2], . . . D[k][p]}. Each of these p+1 points can be used as input to the generator 14 to create gene expression level data, which is the change in gene expression levels in response to the k-th drug (αₖ).

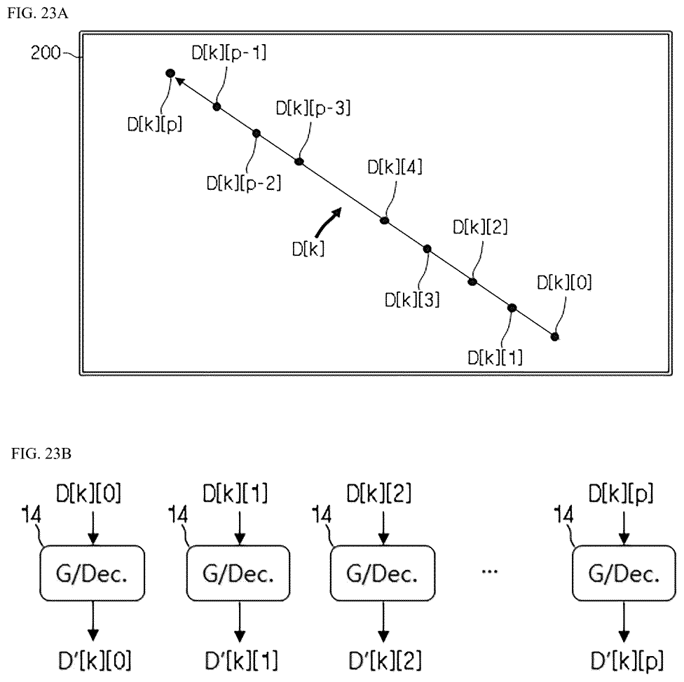

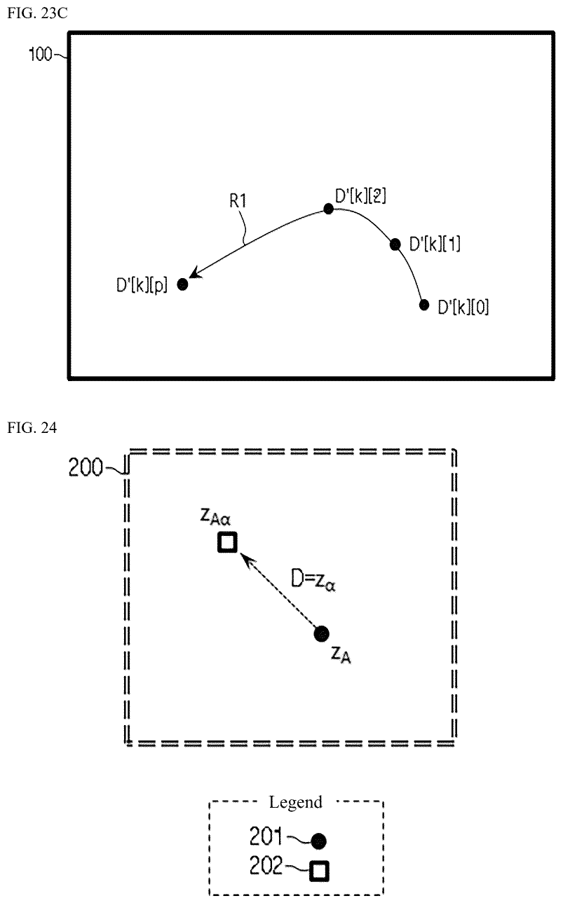

FIG. 23A shows a concept of the vector α′ₖ (=D[k]), representing the effect of the k-th drug (αₖ) defined in the latent space 200, and the p+1 points (D[k][0], D[k][1], D[k][2], . . . D[k][p]) defined in the latent space 200 obtained by dividing the vector D[k] into p equal parts.

FIG. 23B shows a concept of inputting the p+1 points (D[k][0], D[k][1], D[k][2], . . . D[k][p]) into the trained generator 14 to obtain the p+1 reconstructed cell states (D′[k][0], D′[k][1], D′[k][2], . . . D′[k][p]) defined in the cell state space 100 for the k-th drug (αₖ), as explained with reference to FIGS. 19 to 21b.

FIG. 23C shows the p+1 reconstructed cell states (D′[k][0], D′[k][1], D′[k][2], . . . D′[k][p]) mapped in the cell state space 100. The reconstructed cell state (D′[k][0]) corresponds to the cell state of the given cell A in the cell state space 100. The p reconstructed cell states (D′[k][1], D′[k][2], . . . D′[k][p]) represent the transition path (RI) of the given cell A when the k-th drug (αₖ) is administered. As described above, each point in the cell state space 100 represents the gene expression levels of the genes in the cell. The path (RI) represents the change in gene expression levels due to the k-th drug (αₖ).

FIG. 24 is a diagram explaining the meaning of the k-th representative distance vector (D[k]) corresponding to the administration of the k-th drug explained in FIG. 13.

In FIG. 24, the reference symbol D is used instead of D[k] shown in FIG. 13.

The latent space 200 shown in FIG. 24 can be the space generated by the encoder 11 trained using the first loss (Lₜᵣᵢₚₗₑ) and the second loss (L_{delta}) described above. Each point in the latent space 200 can be defined as a vector.

The state of a particular cell A, which has not been treated with a specific drug (α) (i.e., the expression levels of the genes of the specific cell), can be transformed by the encoder 11 into a corresponding point in the latent space 200, which is the latent baseline state 201. The latent baseline state 201 represents the gene expression levels of cell A before the drug (α) is administered. Cell A can vary widely, including normal cells, cancer cells, disease cells with non-cancerous conditions, human cells, or cells from non-human animals or plants.

The reference symbol D represents the vector (state transition vector) in the latent space 200 that indicates the gene expression regulation effect of a given drug (α). That is, the reference symbol D represents the change in the state of an arbitrary cell, i.e., the change in gene expression levels, caused by the administration of the drug (α).

The state of cell A to which the drug (α) has been administered can be transformed by the encoder 11 into the corresponding point in the latent space 200, which is the latent perturbed state 202. The latent perturbed state 202 represents the gene expression levels of cell A when the drug (α) is administered.

The latent baseline state 201 can be considered the initial state of cell A, and the latent perturbed state 202 can be considered the target state of cell A. Here, it can be understood that the drug (α) provides the effect of transitioning the state of cell A from the initial state to the target state.

According to the present invention, the gene expression regulation effect of a specific drug (α) can be expressed as a specific vector in the latent space 200 generated by the encoder 11. This allows the identification of molecular targets for transitioning the initial state of an arbitrary cell to the desired target state. Furthermore, it provides a technology for repositioning the use of a drug known to be effective for treating a specific disease to treat another disease.

In the cell state space 100, defined by the gene expression levels of the cells, the gene expression regulation effect of a specific drug (α) is expressed in different directions depending on the initial state of the specific cell line to which the drug (α) is administered. However, in the latent space 200 generated by the encoder 11, the gene expression regulation effect of a specific drug (α) is expressed as a single fixed vector value regardless of the initial state of the specific cell line. Therefore, there is an advantage in tracking the mechanism of the gene expression regulation effect of the drug (α) based on the latent space 200.

Referring again to FIG. 24, the value of the target state 202, which is the state of the cell perturbed by the drug (α) in the latent space 200, can be observed as being separated into the initial state 201, which is the state of cell A before the drug (α) is administered, and the vector D representing the gene expression regulation effect of the drug (α). As a result, the separated perturbation effects can be applied to “unseen” cells in the latent space 200 to predict the cell response of the “unseen” cells. Here, the target state can be denoted as zag, the initial state as z_{A}, and the gene expression regulation effect of the drug (α) as D=z_{α}.

In FIG. 21B, z^{A}_{α} and z^{B}_{α} each represent the effect of the drug α in the latent space. However, when the encoder 11 is not properly trained, the effect of the drug α in the latent space depends on the cell (A or B) to which the drug α is administered, resulting in different values, so the superscripts A and B are added.

Also, in FIG. 21B, z^{A}_{α} represents the effect of the drug α itself in the latent space, and z_{Aα} represents the state of cell A perturbed by the drug α in the latent space.

In FIG. 24, z_{α} represents the effect of the drug α in the latent space. FIG. 24 illustrates the case where the encoder 11 is properly trained, and Here, the effect of the drug α in the latent space does not depend on the cell (A or B) to which the drug α is administered and maintains a substantially constant value, so the superscripts A and B are not added, and it is simply denoted as z_{α}.

Using the embodiments of the present invention described above, those skilled in the technical field of the present invention can easily make various changes and modifications within the scope that does not depart from the essential characteristics of the present invention. The contents of each claim in the patent claims can be combined with other claims within the scope that can be understood through the specification.

What is claimed is:

1. A method for determining suitability of a k-th drug administered to transition a state of a cell to another state, comprising:
acquiring, by a computing apparatus, a set of baseline states 101, which are data representing states of a set of actual cells, acquiring a k-th set of perturbed states 102, which are data representing transitioned states of the set of actual cells after administering the k-th drug; acquiring an initial cell state 103, which is data representing an initial state of a specific cell line of a first subject; and acquiring a target cell state 104, which is data representing a target state of the specific cell line of the first subject;
encoding, by the computing apparatus, the set of baseline states 101, the k-th set of perturbed states 102, the initial cell state 103, and the target cell state 104 to generate a set of latent baseline states 201, a k-th set of latent perturbed states 202, a latent initial cell state 203, and a latent target cell state 204, respectively;
calculating, by the computing apparatus, a k-th representative distance vector that indicates a distance between the set of latent baseline states and the k-th set of latent perturbed states, and calculating a reference distance vector (DR), which is a distance vector between the latent initial cell state and the latent target cell state; and
calculating, by the computing apparatus, a degree of similarity between the k-th representative distance vector and the reference distance vector to determine suitability of the k-th drug based on the calculated degree of similarity.

2. The method according to claim 1, wherein the higher the degree of similarity, the greater the value of the suitability.

3. The method according to claim 1, wherein the encoder used by the computing apparatus for encoding is an encoder of a VAE (Variational AutoEncoder) trained by a predetermined encoder training unit executed by the computing apparatus, and
wherein the encoder training unit includes a GAN (Generative Adversarial Network) that includes a generator and a discriminator, and
the generator is the decoder of the VAE, and a value output by the encoder of the VAE is input into the generator, and
the encoder training unit is configured to input a state of an arbitrary cell into the encoder to output a latent state, input the output latent state into the generator to output a reconstructed state, and train the encoder, the generator, and the discriminator so that the discriminator determines truthfulness of the reconstructed state based on the state of the arbitrary cell.

4. The method according to claim 3, wherein the method by which the encoder training unit trains the encoder includes:
acquiring a first-first perturbed state x_{Aα}, which is a state of a cell of a first cell line A after administering the first drug α; a first-second perturbed state x_{Bα}, which is a state of a cell of a second cell line B after administering the first drug α; a second-second perturbed state x_{Bβ}, which is a state of the cell of the second cell line after administering a second drug J; and a second-first perturbed state x_{Aβ}, which is a state of the cell of the first cell line (A) after administering the second drug;
independently inputting the first-fist perturbed state x_{Aα}, the first-second perturbed state x_{Bα}, and the second-second perturbed state x_{Bβ} into the encoder to generate a first-first latent perturbed state z_{Aα}, a first-second latent perturbed state z_{Bα}, and a second-second latent perturbed state z_{Bβ}, respectively;
inputting a latent state z′_{Aβ}, which is obtained by adding the second-second latent perturbed state z_{Bβ} to a value obtained by subtracting the first-second latent perturbed state z_{Ba} from the first-first latent perturbed state z_{Aα}, into the generator to generate a second-first reconstructed perturbed state x′_{Aβ}; and
training the encoder using a first loss Lₜᵣᵢₚₗₑ, which utilizes a value obtained by subtracting the second-first perturbed state x_{Aβ} from the second-first reconstructed perturbed state x′_{Aβ}.

5. The method according to claim 3, wherein the method by which the encoder training unit trains the encoder includes:
acquiring a first-first perturbed state x_{Aα}, which is a state of a cell of a first cell line A after administering the first drug α; a first cell state x_{A}, which is a state of a cell of the first cell line; a first-second perturbed state x_{Bα}, which is a state of a cell of a second cell line B after administering the first drug α; and a second cell state x_{B}, which is a state of a cell of the second cell line;
independently inputting the first-first perturbed state x_{Aα}, the first cell state x_{A}, the first-second perturbed state x_{Bα}, and the second cell state x_{B} into the encoder to generate a first-first latent perturbed state z_{Aα}, a first latent cell state z_{A}, a first-second latent perturbed state z_{Bα}, and a second latent cell state z_{B}, respectively;
generating a first perturbation vector z^{A}_{α} by subtracting the first latent cell state z_{A} from the first-first latent perturbed state z_{Aα}; and generating a second perturbation vector z^{B}_{α} by subtracting the second latent cell state z_{B} from the first-second latent perturbed state z_{Bα}; and
training the encoder using a second loss L_{delta}, which utilizes a value obtained by subtracting the second perturbation vector z^{B}_{α} from the first perturbation vector z^{A}_{α}.

6. The method according to claim 3, wherein the method by which the encoder training unit trains the encoder includes:
acquiring a first-first perturbed state x_{Aα}, which is a state of a cell of the first cell line A after administering the first drug α; a first-second perturbed state x_{Ba} which is a state of a cell of a second cell line B after administering the first drug α; a second-second perturbed state x_{Bβ}, which is a state of the cell of the second cell line after administering a second drug β; and a second-first perturbed state x_{Aβ}, which is a state of the cell of the first cell line (A) after administering the second drug; a first cell state x_{A}, which is a state of a cell of the first cell line; a first-second perturbed state x_{Bα}, which is a state of a cell of a second cell line B after administering the first drug α; and a second cell state x_{B}, which is a state of a cell of the second cell line;
independently inputting the first-first perturbed state x_{Aα}, the first-second perturbed state x_{Bα}, the second-second perturbed state x_{Bβ}, the first cell state x_{A}, and the second cell state x_{B} into the encoder to generate a first-first latent perturbed state z_{Aα}, a first-second latent perturbed state z_{Bα}, a second-second latent perturbed state z_{Bβ}, a first latent cell state z_{A}, a first-second latent perturbed state z_{Bα}, and a second latent cell state z_{B}, respectively;
inputting a latent state z′_{Aβ}, which is obtained by adding the second-second latent perturbed state z_{Bβ} to a value obtained by subtracting the first-second latent perturbed state z_{Ba} from the first-first latent perturbed state z_{Aα}, into the generator to generate a second-first reconstructed perturbed state x′_{Aβ}; generating a first perturbation vector z_{Aα} by subtracting the first latent cell state z_{A} from the first-first latent perturbed state z^{A}_{α}; and generating a second perturbation vector z^{B}_{α} by subtracting the second latent cell state z_{B} from the first-second latent perturbed state z_{Bα}; and
training the encoder using a loss L_{G}, which includes a first loss Lₜᵣᵢₚₗₑ, which includes a value obtained by subtracting the second-first perturbed state x_{Aβ} from the second-first reconstructed perturbed state x′_{Aβ}, and a second loss L_{delta}, which includes a value obtained by subtracting the second perturbation vector z^{B}_{α} from the first perturbation vector z^{A}_{α}.

7. The method according to claim 1, wherein the set of baseline states 101, the k-th set of perturbed states 102, the initial cell state 103, and the target cell state 104 are array data consisting of expression levels of the genes in each corresponding cell, defined in a predetermined cell state space 100;
the set of latent baseline states 201, the k-th set of latent perturbed states 202, the latent initial cell state 203, and the latent target cell state 204 are each values defined in a predetermined latent space 200; and
the encoder used by the computing apparatus for encoding is configured to transform values belonging to the cell state space 100 into values belonging to the latent space 200.

8. The method according to claim 3, wherein the set of baseline states 101, the k-th set of perturbed states 102, the initial cell state 103, and the target cell state 104 are array data consisting of expression levels of the genes in each corresponding cell, defined in a predetermined cell state space 100;
the set of latent baseline states 201, the k-th set of latent perturbed states 202, the latent initial cell state 203, and the latent target cell state 204 are each values defined in a predetermined latent space 200; and
the encoder used by the computing apparatus for encoding is configured to transform values belonging to the cell state space 100 into values belonging to the latent space 200,
wherein the method for determining suitability further comprises the steps of:
acquiring a first state, which is a state of a predetermined cell;
transforming the first state into the first latent state defined in the latent space using the encoder;
determining a starting point represented by the first latent state in the latent space, determining an endpoint displaced by the k-th representative distance vector from the starting point, and determining multiple intermediate points on a straight line connecting the starting point and the endpoint; and
inputting, by the trained generator, values of the starting point, the multiple intermediate points, and the endpoint to output reconstructed states belonging to the cell state space 100, corresponding to these values,
wherein the reconstructed states are the states along a transition path of states of the first cell when the k-th drug is administered.

9. The method according to claim 1, wherein the initial state of the specific cell line is a disease state with a particular disease present in a specific cell line, and the target state of the specific cell line is a normal state with the particular disease absent in the specific cell line.

10. The method according to claim 9, wherein the particular disease is cancer.

11. The method according to claim 1, wherein the first subject is a human.

12. The method according to claim 1, wherein the first subject is an animal or a plant other than a human.

13. A computing apparatus comprising: a processing unit and a storage unit, wherein the storage unit contains encoder instruction codes that execute a predetermined encoder and suitability determination instruction codes that execute a method for determining suitability of a k-th drug administered to transition the state of a cell to another state, and wherein the processing unit, by reading and executing the suitability determination instruction codes from the storage unit, performs the steps of
acquiring a set of baseline states, which are data representing states of a set of actual cells, acquiring a k-th set of perturbed states, which are data representing transitioned states of the set of actual cells after administering the k-th drug; acquiring an initial cell state, which is data representing an initial state of a specific cell line of a first subject; and acquiring a target cell state, which is data representing a target state of the specific cell line of the first subject;
encoding the set of baseline states, the k-th set of perturbed states, the initial cell state, and the target cell state 104 to generate a set of latent baseline states, a k-th set of latent perturbed states, a latent initial cell state, and a latent target cell state, respectively;
calculating a k-th representative distance vector that indicates a distance between the set of latent baseline states and the k-th set of latent perturbed states, and calculating a reference distance vector, which is a distance vector between the latent initial cell state and the latent target cell state; and
calculating a degree of similarity between the k-th representative distance vector and the reference distance vector to determine suitability of the k-th drug based on the calculated degree of similarity.

14. The computing apparatus according to claim 13, the processing unit is configured to independently and repeatedly execute the suitability determination instruction codes for multiple different administered drugs and to determine the suitability ranking of the multiple administered drugs with reference to the multiple suitabilities determined for the multiple different administered drugs.

15. The computing apparatus according to claim 14, wherein the encoder used by the computing apparatus for encoding is an encoder of a VAE (Variational AutoEncoder) trained by a predetermined encoder training unit executed by the computing apparatus, and
wherein the encoder training unit includes a GAN (Generative Adversarial Network) that includes a generator and a discriminator, and
the generator is the decoder of the VAE, and a value output by the encoder of the VAE is input into the generator, and
the encoder training unit is configured to input a state of an arbitrary cell into the encoder to output a latent state, input the output latent state into the generator to output a reconstructed state, and train the encoder, the generator, and the discriminator so that the discriminator determines truthfulness of the reconstructed state based on the state of the arbitrary cell;
wherein the method by which the encoder training unit trains the encoder includes:
acquiring a first-first perturbed state x_{Aα}, which is a state of a cell of a first cell line A after administering the first drug α; a first-second perturbed state x_{Bα}, which is a state of a cell of a second cell line B after administering the first drug α; a second-second perturbed state x_{Bβ}, which is a state of the cell of the second cell line after administering a second drug β; and a second-first perturbed state x_{Aβ}, which is a state of the cell of the first cell line (A) after administering the second drug;
independently inputting the first-fist perturbed state x_{Aα}, the first-second perturbed state x_{Bα}, and the second-second perturbed state x_{Bβ} into the encoder to generate a first-first latent perturbed state z_{Aα}, a first-second latent perturbed state z_{Bα}, and a second-second latent perturbed state z_{Bβ}, respectively;
inputting a latent state a′_{Aβ}, which is obtained by adding the second-second latent perturbed state z_{Bβ} to a value obtained by subtracting the first-second latent perturbed state z_{Ba} from the first-first latent perturbed state z_{Aα}, into the generator to generate a second-first reconstructed perturbed state x′_{Aβ}; and
training the encoder using a first loss Lₜᵣᵢₚₗₑ, which utilizes a value obtained by subtracting the second-first perturbed state x_{Aβ} from the second-first reconstructed perturbed state x′_{Aβ}.

16. A non-volatile storage medium readable by a computing apparatus, wherein the non-volatile storage medium stores a software program, wherein the software program records suitability determination instruction codes for executing a method for determining suitability of a k-th drug administered to transition a state of a cell to another state, wherein the suitability determination instruction codes allow the computing apparatus to perform the steps of:
acquiring a set of baseline states, which are data representing states of a set of actual cells, acquiring a k-th set of perturbed states, which are data representing transitioned states of the set of actual cells after administering the k-th drug; acquiring an initial cell state, which is data representing an initial state of a specific cell line of a first subject; and acquiring a target cell state, which is data representing a target state of the specific cell line of the first subject;
encoding the set of baseline states, the k-th set of perturbed states, the initial cell state, and the target cell state 104 to generate a set of latent baseline states, a k-th set of latent perturbed states, a latent initial cell state, and a latent target cell state, respectively;
calculating a k-th representative distance vector that indicates a distance between the set of latent baseline states and the k-th set of latent perturbed states, and calculating a reference distance vector, which is a distance vector between the latent initial cell state and the latent target cell state; and
calculating a degree of similarity between the k-th representative distance vector and the reference distance vector to determine suitability of the k-th drug based on the calculated degree of similarity.
